(19) United States
(12) Patent Application Publication
(10) Pub. No.: US 2005/0143622 A1
(43) Pub. Date: Jun. 30, 2005

(54) Medical examination device having patient positioning means movable under safety control

(76) Inventor: Matheus Jakobus Maria Evers, Eindhoven (NL)

Correspondence Address: Thomas M Lundin;Philips Intellectual Property & Standards, 595 Miner Road, Cleveland, OH, 44143, US

(21) Appl. No.: 10/510,851

(22) PCT Filed: Apr. 1, 2003
(86) PCT No.: PCT/IB03/01373

(30) Foreign Application Priority Data
Apr. 18, 2002 (EP) 02076523.6

(51) Int. Cl. A61B 1/04

(52) U.S. Cl. 600/109

(57) ABSTRACT

The invention refers to a medical examination device (1) that comprises means (3) for producing medical images of a patient to be examined. The patient is positioned on positioning means (4) that are movable relative to the imaging means (3) by moving means (6) under safety control. The examination device thereto comprises means (8) for determining the presence of obstacles in or near the examination device that are arranged to control the moving means (6).

The present invention relates to a medical examination device, comprising means for producing medical images of a patient to be examined, means for positioning the patient and means for (semi-) automatically moving the positioning means relative to the imaging means.

Many varieties of such a medical examination device are known in practice. One example thereof is a Magnetic Resonance Imaging (MRI) apparatus. The known MRI apparatus comprises a housing that accommodates magnets for producing medical images of a patient to be examined. In addition thereto a ridable table or trolley is provided on which the patient is to be positioned, that is automatically movable through a passage in the housing.

It is a tendency in the relevant field to provide an MRI apparatus with a more open character resulting in a larger passage in the housing. As a result of this tendency the patient positioning means have also been further developed and are now arranged to not only move in longitudinal direction, but also in transverse direction or even in a combination thereof.

The disadvantage of such an open MRI apparatus is that any obstacle in the path of the movable trolley might disturb the examination and might even endanger the patient or the medically skilled personnel attending the examination. For instance, lose hanging cables of medical equipment might get jammed between the trolley and the housing, leading to possible equipment failure. It is even conceivable that body parts of the patient or members of the medically skilled personnel might get trapped between the trolley and the housing leading to possible injuries.

It is an object of the invention to provide a medical examination device of the type as described above that solves this problem.

The medical examination device according to the invention is therefore characterised in that the examination device further comprises presence determining means for determining the presence of objects in or near the examination device, which presence determining means are arranged to control the moving means. By controlling the moving means on the basis of information about the presence of objects in the vicinity of the examination device the safety of the patient and medical personnel can be guaranteed.

It is mentioned that DE 197 25 137 describes a medical examination device, comprising means for producing medical images of a patient to be examined, means for positioning the patient and means for determining the position of objects that are present in or near the examination device. Contrary to the device according to the invention the known device is a static device comprising a C bow apparatus for imaging a patient on a static table. The position of the patient, the patient table and the C bow apparatus are monitored in order to correct image artefacts arising form variations in those positions.

In a preferred embodiment of the device according to the invention the examination device further comprises a control unit that is connected to the presence determining means and to the moving means, wherein the control unit is arranged to check whether a number of predetermined safety criteria are met and to control the moving means based on the outcome of said check. All relevant safety criteria can be input in the control device. Advantageously any changes in the safety criteria can be realised by adjusting the control unit. This is especially easily to accomplish when the control unit is made programmable.

According to a further preferred embodiment the presence determining means are arranged to cover one or more areas in or near the examination device and the control unit is arranged to check whether said areas are free of obstacles. Using this embodiment certain areas in the vicinity of the device can be defined that and appropriate safety criteria can be defined in order to assure that these areas are kept free of obstacles during examination.

In yet a further preferred embodiment the control unit is arranged to stop the moving means from moving the positioning means if one or more of the predetermined safety criteria are not met. This embodiment provides optimal protection to the persons involved in the examination.

In another embodiment the presence determining means are arranged to determine the position of objects present in or near the examination device. Based on the position information appropriate action can be undertaken with respect to controlling the moving means.

In a further detailed embodiment thereof the presence determining means are arranged to determine the position of the patient and to provide said patient position to the control unit for use in producing the medical images. Accurate information about the patient's position is crucial for producing correct images. This becomes even more important when the positioning means are movable in different directions, as is the case in the new generation of open MRI apparatus. A real time correction of the position of the imaging means is possible in another embodiment, wherein the control unit is arranged to control the imaging means based on the actual position of the patient. An afterwards correction of the images is possible in yet another embodiment, wherein the control unit is arranged to correct the medical images based on the actual position of the patient during recording thereof.

The position of the presence determining means is optimal when the presence determining means are located on the positioning means. In case the examination device comprises a housing having a passage for the patient, the position of the presence determining means is further optimised when the presence determining means are located on the housing.

The function of the presence determining means is best performed by means of ultrasonic equipment.

The invention is very suitable for use in a Magnetic Resonance Imaging (MRI) apparatus.

The invention will be further explained by means of the attached drawing, in which:

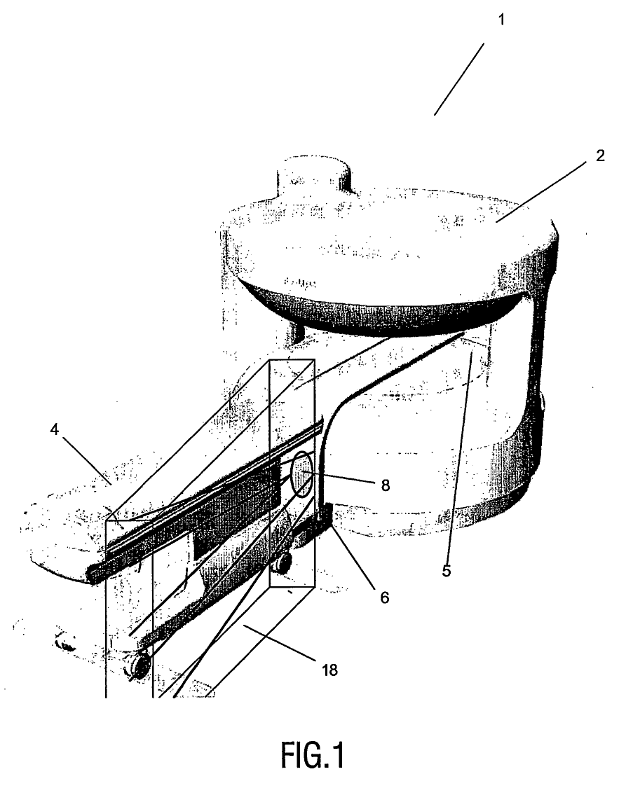

FIG. 1 schematically shows a first embodiment of the medical examination device according to the invention;

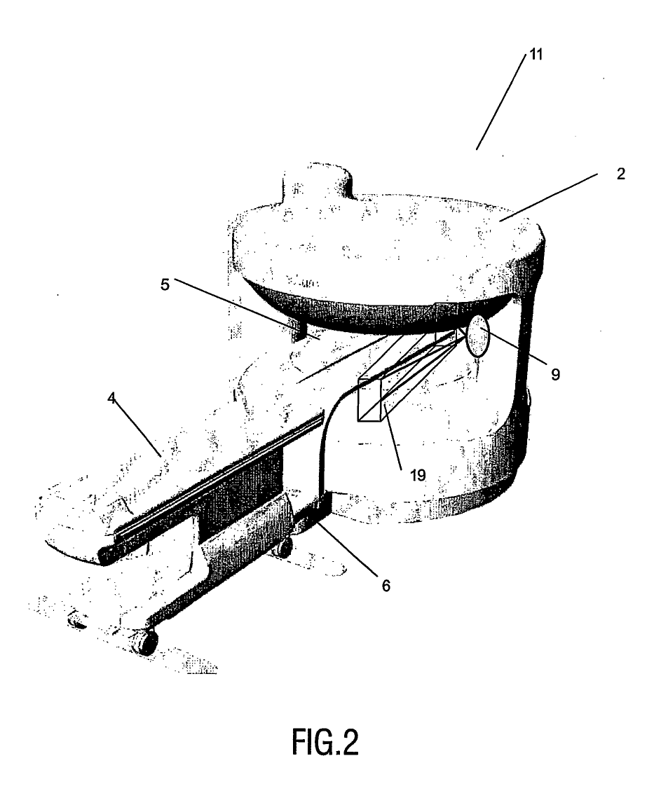

FIG. 2 schematically shows a second embodiment of the medical examination device according to the invention;

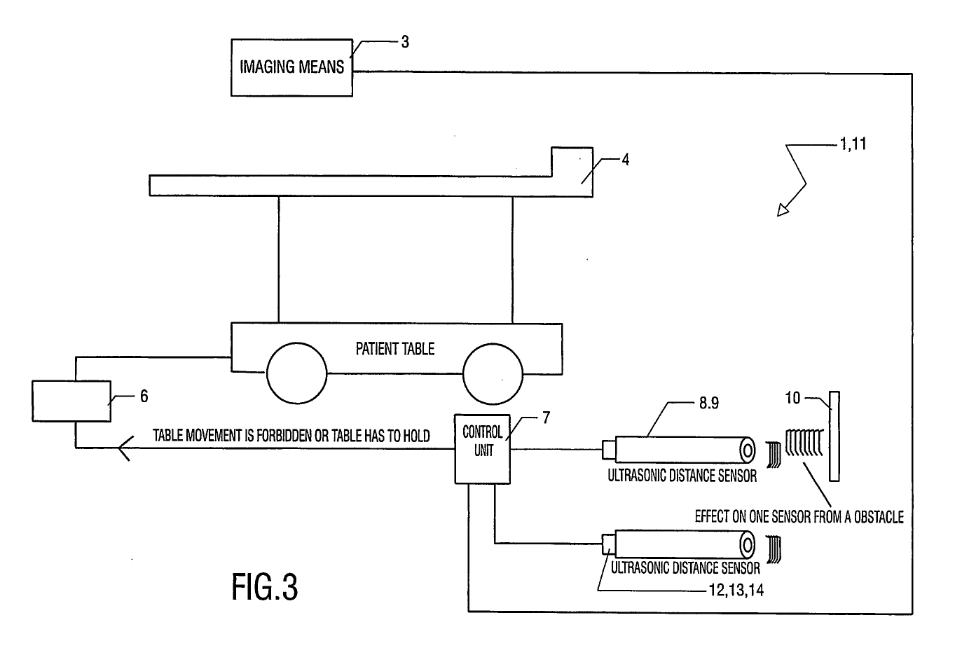

FIG. 3 shows a block diagram of the components of the medical examination device according to the invention.

In all figs. equal objects are denoted with equal reference numerals.

FIG. 1 schematically shows a medical examination device 1 according to the invention. In the preferred embodiment shown the medical examination device I is an MRI apparatus. MRI apparatus 1 comprises a housing 2, in which means 3 (see FIG. 3) for producing medical images of a patient to be examined are incorporated.

Medical examination device 1 is provided with means for positioning the patient. The positioning means comprise a ridable table or trolley 4 on which the patient is to be laid. Trolley 4 is (semi-) automatically movable through a passage 5 in the housing 2 by moving means 6 under the control of a control unit 7 (see FIG. 3).

An MRI apparatus 1 as described so far is known in the art and will therefore not be discussed in more detail in the context of this application.

FIG. 2 schematically shows a second embodiment 11 of the medical examination device according to the invention. FIG. 3 shows a block diagram of the components of both embodiments 1 and 11 of the medical examination device according to the invention.

According to the invention the medical examination device 1, 11 is provided with means 8, 9 respectively for determining the presence of objects 10 that at are present in or near the examination device 1. These presence determining means 8, 9 are arranged to cover one or more areas 18, 19 in or near the examination device 1. Preferably the presence determining means comprise ultrasonic sensors. Ultrasonic sensor 8 is positioned on the trolley 4 and is able to detect the presence of objects in the area 18 along the corresponding side of the trolley. Ultrasonic sensor 9 is positioned on the housing 2 and is able to detect the presence of objects in the area 19 extending in the passage 2 at one side of the housing. Many types of ultrasonic sensors that are suitable for the above-described use are known per se in the relevant technical field.

In the preferred embodiment illustrated the presence determining means 8,9 are arranged to control the moving means 6. Thereto a control unit 7 is connected to the presence determining means 8, 9 as well as to the moving means. The control unit receives information from the presence determining means 8, 9 about the presence of any objects 10 that at are present in or near the examination device 1. In the embodiment shown the ultrasonic sensors 8, 9 provide signals relating to the presence of an object 10 in the areas 18 and/or 19. Based on that information the control unit is arranged to check whether a number of predetermined safety criteria are met and to control the moving means 6 based on the outcome of said check. The control unit may comprise to that end suitable hardware and/or software that is per se available in the relevant technical field.

Various safety criteria may be implemented depending on the environment in which the medical examination device is functioning. For instance one safety criterion may include that certain areas to be checked should be free of obstacles to assure the safety of the patient and the personnel. In the situation in FIG. 3 the presence of the object(s) 10 will be detected by the ultrasonic sensors 8, 9. Object(s) 10 may be components of equipment, such as cables, wires, tubes or the like, and/or human(s) and/or human body parts. This information will be send to the control unit 7 and the check on that safety criterion will fail. Based thereon the control unit 7 is arranged to control the moving means 6. The control unit may for instance be arranged to stop the moving means 6 from moving the positioning means 4 if one or more of the predetermined safety criteria are not met. In the embodiment shown, wherein the safety criterion that areas 18, 19 should be free of obstacles is not met, the table 4 is stopped.

It is noted that many other practical safety criteria can be implemented. As an alternative the control unit may for instance be arranged to slow down the moving means 6 during moving the positioning means 4, if one or more of the predetermined safety criteria are not met In addition to stopping or slowing down the moving means 6 (or as another alternative thereto) the control unit may for instance be arranged to alert the persons present and to produce an alarm signal, such as an alarm sound and/or an alarm light.

Preferably the presence determining means 8, 9 are arranged to determine the position of objects present in or near the examination device. Ultrasonic equipment that is suitable therefore, such as ultrasonic distance sensors, is available in the field. Based on the position information appropriate action can then be undertaken with respect to controlling the moving means 6. Based on the distance from the positioning means 4 to the object 10 the moving means can for instance be slowed down or stopped and/or an alarm signal can be given.

In an even more sophisticated embodiment the presence determining means 8, 9 are arranged to determine the position of the patient. The patient position is supplied to the control unit for use in producing the medical images. During an MRI examination the position of the patient changes. The moving means 6 move the patient with respect to the imaging means 3. In addition thereto the patient may himself inadvertently move during the examination. It is therefore known to monitor the position of the patient during the examination in order to produce correct medical images wherein the actual position of the patient is taken into account.

In the embodiment shown additional ultrasonic sensors 12, 13, 14 are arranged to determine the x, y and z coordinates of the actual position of the patient during the examination. The actual patient position is supplied to the control unit and is used to control the imaging means 3. The scanning components of the imaging means are adjusted to the actual position of the patient. As an alternative the control unit 7 may be arranged to record the various patient positions during the examination and to correct the medical images afterwards to the actual position of the patient during the recording thereof. In general the device according to the invention refers to a medical examination device having patient positioning means that are movable under safety control. Although the invention is herein described in the context of a MRI apparatus, it will be clear to any person skilled in the art that the invention is suitable for use in any medical examination device having positioning means for moving the patient relative to the imaging means. An example thereof is X-ray devices in general, and Computed Tomography devices in particular.

The invention is thus of course not limited to the described or shown embodiments, but generally extends to any embodiment, which falls within the scope of the appended claims as seen in light of the foregoing description and drawings.

1. Medical examination device comprising means for producing medical images of a patient to be examined, means for positioning the patient and means for (semi-) automatically moving the positioning means relative to the imaging means, characterized in that, the examination device further comprises presence determining means for determining the presence of objects in or near the examination device, which presence determining means are arranged to control the moving means.

2. Medical examination device according to claim 1, wherein the examination device further comprises a control unit that is connected to the presence determining means and to the moving means wherein the control unit is arranged to check whether a number of predetermined safety criteria are met and to control the moving means based on the outcome of said check.

3. Medical examination device according to claim 2, wherein the presence determining means are arranged to cover one or more areas in or near the examination device and wherein the control unit is arranged to check whether said areas are free of obstacles.

4. Medical examination device according to claim 2, wherein the control unit is arranged to stop the moving means from moving the positioning means if one or more of the predetermined safety criteria are not met.

5. Medical examination device according to claim 1, wherein the presence determining means are arranged to determine the position of objects present in or near the examination device.

6. Medical examination device according to claim 5, wherein the presence determining means are arranged to determine the position of the patient and to provide said patient position to the control unit for use in producing the medical images.

7. Medical examination device according to claim 6, wherein the control unit is arranged to control the imaging means based on the actual position of the patient.

8. Medical examination device according to claim 6, wherein the control unit is arranged to correct the medical images based on the actual position of the patient.

9. Medical examination device according to claim 1, wherein the presence determining means are located on the positioning means.

10. Medical examination device according to claim 1, wherein the examination device comprises a housing having a passages for the patient, wherein the presence determining means are located on the housing.

11. Medical examination device according to claim 1 wherein the presence determining means comprise ultrasonic equipment.

12. Medical examination device according to claim 1 wherein the examination device is a Magnetic Resonance Imaging (MRI) apparatus.
